(19) United States
(12) Patent Application Publication
(10) Pub. No.: US 2002/0197658 A1
(43) Pub. Date: Dec. 26, 2002

(54) Cancer associated protein kinase and its use

(76) Inventors: Allen Delaney, Vancouver (CA); Thillainathan Yoganathan, Richmond (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/143,133

(22) Filed: May 9, 2002

Related U.S. Application Data
(60) Provisional application No. 60/290,555, filed on May 10, 2001.

(51) Int. Cl. C12Q 1/68; G01N 33/574; A61K 31/00

(52) U.S. Cl. 435/7.23; 800/10; 435/6; 514/1

(57) ABSTRACT

Detection of expression of the provided protein kinase in cancers is useful as a diagnostic, for determining the effectiveness of drugs, and determining patient prognosis. The encoded polypeptides further provides a target for screening pharmaceutical agents effective in inhibiting the growth or metastasis of tumor cells.

### BACKGROUND OF THE INVENTION

An accumulation of genetic changes underlies the development and progression of cancer, resulting in cells that differ from normal cells in their behavior, biochemistry, genetics, and microscopic appearance. Mutations in DNA that cause changes in the expression level of key proteins, or in the biological activity of proteins, are thought to be at the heart of cancer. For example, cancer can be triggered in part when genes that play a critical role in the regulation of cell division undergo mutations that lead to their over-expression. “Oncogenes” are involved in the dysregulation of growth that occurs in cancers.

Oncogene activity may involve protein kinases, enzymes that help regulate many cellular activities, particularly signaling from the cell membrane to the nucleus to initiate the cell's entrance into the cell cycle and to control other functions.

Oncogenes may be tumor susceptibility genes, which are typically up-regulated in tumor cells, or may be tumor suppressor genes, which are down-regulated or absent in tumor cells. Malignancies can arise when a tumor suppressor is lost and/or an oncogene is inappropriately activated. When such mutations occur in somatic cells, they result in the growth of sporadic tumors.

Hundreds of genes have been implicated in cancer, but in most cases relationships between these genes and their effects are poorly understood. Using massively parallel gene expression analysis, scientists can now begin to connect these genes into related pathways.

Phosphorylation is important in signal transduction mediated by receptors via extracellular biological signals such as growth factors or hormones. For example, many oncogenes are protein kinases, i.e. enzymes that catalyze protein phosphorylation reactions or are specifically regulated by phosphorylation. In addition, a kinase can have its activity regulated by one or more distinct protein kinases, resulting in specific signaling cascades.

Cloning procedures aided by homology searches of EST databases have accelerated the pace of discovery of new genes, but EST database searching remains an involved and onerous task. More than 1.6 million human EST sequences have been deposited in public databases, making it difficult to identify ESTs that represent new genes. Compounding the problems of scale are difficulties in detection associated with a high sequencing error rate and low sequence similarity between distant homologues.

MAPKs have been shown to mediate multiple cellular pathways regulating growth and differentiation. Despite a long-felt need to understand and discover methods for regulating cells involved in various disease states, the complexity of signal transduction pathways has been a barrier to the development of products and processes for such regulation. Accordingly, there is a need in the art for improved methods for detecting and modulating the activity of MAPKs, and for treating diseases associated with the MAPK signal transduction pathway.

Relevant Literature

The use of genomic sequence in data mining for signaling proteins is discussed in Schultz et al. (2000) Nature Genetics 25:201. The MAPK protein family has been reviewed, for example by Meskiene I, and Hirt, H. (2000) Plant Mol Biol 42(6):791-806. MAP3K has been discussed, for example, by Ing, Y. L. et al. (1994) Oncogene. 9: 1745-1750 and also by Courseaux, A. e.al. (1996) Genomics, 37:354-365 Serine/threonine protein kinases have been reviewed, for example, by Cross T G, et al.( 2000) Exp Cell Res. Apr 10;256(1):34-41. The gene accession number for EST clone K91 is AI803752.

### SUMMARY OF THE INVENTION

MAP3K11 protein is shown to be over-expressed in cancer cells. Detection of MAP3K11 expression in cancers is useful as a diagnostic, for determining the effectiveness of, and mechanism of action for, potential new drugs, and for determining patient prognosis. MAP3K11 further provides a target for screening pharmaceutical agents effective in inhibiting the growth or metastasis of tumor cells. In one embodiment of the invention, a complete nucleotide sequence of human Map3K11 is provided.

### DESCRIPTION OF THE SPECIFIC EMBODIMENTS

Methods are provided for determining whether cells in a sample are cancerous. MAP3K11 is shown to be over-expressed in cancer cells. Detection of MAP3K11 over-expression in cancers provides a useful diagnostic for predicting patient prognosis and probability of drug effectiveness. Generally the amount of MAP3K11 detected will be compared to negative control samples from normal tissue or from known tumor cells. The presence of increased levels of MAP3K11 specific binding is indicative of a an MAP3K11 associated tumor, usually at least about a 2 fold increase will be taken as a positive reaction.

MAP3K11 provides a target for drug screening or altering expression levels, and for determining other molecular targets involved in the kinase signal transduction pathways involved in transformation and growth of tumor cells.

The human gene sequence encoding MAP3K11, is provided as SEQ ID NO:1, and the encoded polypeptide product is provided as SEQ ID NO: 2. The MAP3K11 genetic sequence is isolated and obtained in substantial purity, generally as other than an intact chromosome. Usually, the DNA will be obtained substantially free of other nucleic acid sequences that do not include an MAP3K11 gene sequence or fragment thereof, generally being at least about 50%, usually at least about 90% pure and is typically “recombinant”, i.e. flanked by one or more nucleotides with which it is not normally associated on a naturally occurring chromosome. Dot blot analysis of probes prepared from mRNA of tumors showed that expression of MAP3K11 is consistently up-regulated in clinical samples of human tumors.

Many of the transduction pathways in mammalian cells that involve the sequential activation of a series of signaling proteins linking the cell surface with nuclear targets are mediated by mitogen-activated protein kinases (MAPKs) (also called extracellular signal-regulated kinases or ERKs). In mammalian cells, three parallel MAPK pathways have been described. Generally, MAPKs are rapidly activated in response to ligand binding by both growth factor receptors that are tyrosine kinases (such as the EGF receptor) and receptors that are coupled to G proteins. Phosphorylation of tyrosine residues leads to generation of docking sites for SH2 (Src homology 2) and PTB (phosphotyrosine binding) domains of adaptor proteins. (see Lemmon et al. (1994) Trends Biochem Sci 19:459-63; and Pawson et al. (1997) Science 278:2075-80.

Mitogen-activated protein (MAP) kinases include extracellular signal-regulated protein kinase (ERK), c-Jun amino-terminal kinase (JNK), and p38 subgroups. These MAP kinase isoforms are activated by dual phosphorylation on threonine and tyrosine (Derijard el al. (1995) Science 267(5198):682-5). MAP3K11 is an isoform that has been described by Ing et al. (1994) Oncogene 9:1745-1750. It has been mapped via fluorescence in situ hybridization to 11q13.1-q13.3 (Courseaux el al. (1996) Genomics 37:354-365). MAP3K also shares homology, including an unusual leucine zipper-basic motif, with a family of protein kinases known as mixed lineage protein kinases.

Ing et al. (supra.) found that MAP3K contains an SH3 domain and has a long carboxy-terminal tail that exhibits proline rich motifs similar to known SH3 binding sites. SH3 domains play the role of a protein switch, which is turned on by a number of receptor-mediated signals to which it responds by changes in kinase activity and by changes in intracellular localization. It acts as part of an adapter molecule and recruits downstream proteins in a signaling pathway.

Diagnostic Methods

Determination of the presence of MAP3K11 is used in the diagnosis, typing and staging of tumors. Detection of the presence of MAP3K11 is performed by the use of a specific binding pair member to quantitate the specific protein, DNA or RNA present in a patient sample. Generally the sample will be a biopsy or other cell sample from the tumor. Where the tumor has metastasized, blood samples may be analyzed.

Specific Binding Members

In a typical assay, a tissue sample, e.g. biopsy, blood sample, etc. is assayed for the presence of MAP3K11 specific sequences by combining the sample with a MAP3K11 specific binding member, and detecting directly or indirectly the presence of the complex formed between the two members. The term “specific binding member” as used herein refers to a member of a specific binding pair, i.e. two molecules where one of the molecules through chemical or physical means specifically binds to the other molecule. In this particular case one of the molecules is MAP3K11, where the term MAP3K11 is intended to include any protein substantially similar to the amino acid sequence provided in SEQ ID NO:2, or a fragment thereof; or any nucleic acid substantially similar to the nucleotide sequence provided in SEQ ID NO:1, or a fragment thereof. The complementary members of a specific binding pair are sometimes referred to as a ligand and receptor.

Binding pairs of interest include antigen and antibody specific binding pairs, peptide-MHC antigen and T cell receptor pairs; complementary nucleotide sequences (including nucleic acid sequences used as probes and capture agents in DNA hybridization assays); kinase protein and substrate pairs; autologous monoclonal antibodies, and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Nucleic acid sequences. In another embodiment of the invention, nucleic acids are used as a specific binding member. Sequences for detection are complementary to a MAP3K11 sequence. The nucleic acids of the invention include nucleic acids having a high degree of sequence similarity or sequence identity to SEQ ID NO:1. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to SEQ ID NO:1 under stringent hybridization conditions.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in tumor cells.

Probes specific to the nucleic acid of the invention can be generated using the nucleic acid sequence disclosed in SEQ ID NO:1. The probes are preferably at least about 18 nt, 25 nt, 50 nt or more of the corresponding contiguous sequence of SEQ ID NO:1, and are usually less than about 2, 1, or 0.5 kb in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

Antibodies. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a polypeptide, particularly MAP3K11.

As used herein, a compound which specifically binds to human protein MAP3K11 is any compound (such as an antibody) which has a binding affinity for any naturally occurring isoform, splice variant, or polymorphism. As one of ordinary skill in the art will appreciate, such “specific” binding compounds (e.g., antibodies) may also bind to other closely related proteins which exhibit significant homology, for example, having greater than 90% identity, more preferably greater than 95% identity, and most preferably greater than 99% identity with the amino acid sequence of SEQ ID NO:2. Such proteins may include truncated forms or domains of SEQ ID NO:2, and recombinantly engineered alterations of SEQ ID NO:2. For example, a portion of SEQ ID NO:2 may be engineered to encode a non-naturally occurring cysteine for cross-linking to an immunoconjugate protein, as described below.

Selection of antibodies which alter (enhance or inhibit) the binding of a compound to MAP3K11 may be accomplished by a straightforward binding inhibition/enhancement assay. According to standard techniques, the binding of a labeled (e.g., fluorescently or enzyme-labeled) antibody to a protein of the invention, which has been immobilized in a microtiter well, is assayed using standard phosphatase assays in both the presence and absence of the ligand. The change in binding is indicative of either an enhancer (increased binding) or competitive inhibitor (decreased binding) relationship between the antibody and the ligand. Such assays may be carried out in high-throughput formats (e.g., 384 well plate formats, in robotic systems) for the automated selection of monoclonal antibody candidates for use as ligand or substrate-binding inhibitors or enhancers.

In addition, antibodies that are useful for altering the function of a protein of the invention may be assayed in functional formats. In cell-based assays of activity, expression of a protein of the invention is first verified in the particular cell strain to be used. If necessary, the cell line may be stably transfected with a coding sequence under the control of an appropriate constituent promoter, in order to express a protein of the invention at a level comparable to that found in primary tumors. The ability of the tumor cells to survive in the presence of the candidate function-altering -antibody is then determined. Similarly, in vivo models for human cancer, particularly colon, pancreas, lung and ovarian cancer are available as nude mice/SCID mice or rats, have been described. Once expression of a protein of the invention in the tumor model is verified, the effect of the candidate antibodies on the tumor masses in these models can evaluated, wherein the ability of the antibody candidates to alter phosphatase activity is indicated by a decrease in tumor growth or a reduction in the tumor mass. Thus, antibodies that exhibit the appropriate anti-tumor effect may be selected without direct knowledge of a binding ligand.

Generally, as the term is utilized in the specification, “antibody” or “antibody moiety” is intended to include any polypeptide chain-containing molecular structure that has a specific shape which fits to and recognizes an epitope, where one or more non-covalent binding interactions stabilize the complex between the molecular structure and the epitope. Antibodies which bind specifically to a protein of the invention are referred to as anti-phosphatase antibodies. The specific or selective fit of a given structure and its specific epitope is sometimes referred to as a “lock and key” fit. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinantly, and may be modified to reduce their antigenicity.

Polyclonal antibodies may be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, an antigenic portion of a MAP3K11 polypeptide is initially injected into any of a wide variety of mammals (e.g., mice, rats, rabbits, sheep or goats). Alternatively, in order to generate antibodies to relatively short peptide portions of MAP3K11, a superior immune response may be elicited if the polypeptide is joined to an immunogenic carrier, such as ovalbumin, BSA, KLH, pre-S HBsAg, other viral or eukaryotic proteins, and the like. The peptide-conjugate is injected into the animal host, preferably according to a predetermined schedule incorporating one or more booster immunizations, and the animals are bled periodically. Polyclonal antibodies specific for the polypeptide may then be purified from such anti-sera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine phosphatase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, Nature 1975 256:495 (the disclosure of which is herein incorporated by reference).

Large quantities of monoclonal antibodies from the secreting hybridomas may then be produced by injecting the clones into the peritoneal cavity of mice and harvesting the ascites fluid therefrom. The mice, preferably primed with pristine, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column chromatography or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant. It is preferred that such antibodies by humanized or chimerized according to one of the procedures outlined below.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Preferably, recombinant antibodies are produced in a recombinant protein production system which correctly glycosylates and processes the immunoglobulin chains, such as insect or mammalian cells, as is known in the art.

Antibodies that have a reduced propensity to induce a violent or detrimental immune response in humans (such as anaphylactic shock), and which also exhibit a reduced propensity for priming an immune response which would prevent repeated dosage with the antibody therapeutic or imaging agent (e.g., the human-anti-murine-antibody “HAMA” response), are preferred for use in the invention. Although some increased immune response against the tumor is desirable, the concurrent binding and inactivation of the therapeutic or imaging agent generally outweighs this benefit. Thus, humanized, chimeric, or xenogenic human antibodies, which produce less of an immune response when administered to humans, are preferred for use in the present invention.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference.) Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

Alternatively, polyclonal or monoclonal antibodies may be produced from animals which have been genetically altered to produce human immunoglobulins, such as the Abgenix XenoMouse or the Medarex HuMAb® technology. The transgenic animal may be produced by initially producing a “knock-out” animal which does not produce the animal's natural antibodies, and stably transforming the animal with a human antibody locus (e.g., by the use of a human artificial chromosome.) Only human antibodies are then made by the animal. Techniques for generating such animals, and deriving antibodies therefrom, are described in U.S. Pat. Nos. 6,162,963 and 6,150,584, incorporated fully herein by reference.

Alternatively, single chain antibodies (Fv, as described below) can be produced from phage libraries containing human variable regions (described in e.g. U.S. Pat. No. 6,174,708, incorporated fully herein by reference).

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Fv fragments are heterodimers of the variable heavy chain domain (V_{H}) and the variable light chain domain (V_{L}). The heterodimers of heavy and light chain domains that occur in whole IgG, for example, are connected by a disulfide bond. Recombinant Fvs in which V_{H} and V_{L} are connected by a peptide linker are typically stable, see, for example, Huston et al., Proc Natl Acad Sci USA (1988) 85:5879-5883 and Bird et al., Science (1988) 242:423-426, both fully incorporated herein, by reference. These are single chain Fvs which have been found to retain specificity and affinity and have been shown to be useful for imaging tumors and to make recombinant immunotoxins for tumor therapy. However, researchers have found that some of the single chain Fvs have a reduced affinity for antigen and the peptide linker can interfere with binding. Improved Fv's have also been made which comprise stabilizing disulfide bonds between the V_{H} and V_{L} regions, as described in U.S. Pat. No. 6,147,203, incorporated fully herein by reference. Any of these minimal antibodies may be utilized in the present invention, and those which are humanized to avoid HAMA reactions are preferred for use in embodiments of the invention.

In addition, derivatized immunoglobulins with added chemical linkers, detectable moieties (fluorescent dyes, enzymes, substrates, chemiluminescent moieties), or specific binding moieties (such as streptavidin, avidin, or biotin) may be utilized in the methods and compositions of the present invention. For convenience, the term “antibody” or “antibody moiety” will be used throughout to generally refer to molecules which specifically bind to a MAP3K11 epitope, although the term will encompass all immunoglobulins, derivatives, fragments, recombinant or engineered immunoglobulins, and modified immunoglobulins, as described above.

Candidate anti-phosphatase antibodies can be tested for activity by any suitable standard means. As a first screen, the antibodies may be tested for binding against the antigen utilized to produce them, or against the entire extracellular domain or protein. As a second screen, candidates may be tested for binding to an appropriate cell line, or to primary tumor tissue samples. For these screens, the candidate antibody may be labeled for detection (e.g., with fluorescein or another fluorescent moiety, or with an enzyme such as horseradish peroxidase). After selective binding is established, the candidate antibody, or an antibody conjugate produced as described below, may be tested for appropriate activity (i.e., the ability to decrease tumor cell growth and/or to aid in visualizing tumor cells) in an in vivo model, such as an appropriate cell line, or in a mouse or rat or mouse tumor model, as described above.

Methods for Quantitation of Nucleic Acids

Nucleic acid reagents derived from the sequence of SEQ ID NO:1 are used to screen patient samples, e.g. biopsy-derived tumors, inflammatory samples such as arthritic synovium, etc., for amplified DNA in the cell, or increased expression of the corresponding mRNA or protein. DNA-based reagents are also designed for evaluation of chromosomal loci implicated in certain diseases e.g. for use in loss-of-heterozygosity (LOH) studies, or design of primers based on coding sequences.

The polynucleotides of the invention can be used to detect differences in expression levels between two cells, e.g., as a method to identify abnormal or diseased tissue in a human. The tissue suspected of being abnormal or diseased can be derived from a different tissue type of the human, but preferably it is derived from the same tissue type; for example, an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon, etc.) A difference between the polynucleotide-related gene, mRNA, or protein in the two tissues which are compared, for example, in molecular weight, amino acid or nucleotide sequence, or relative abundance, indicates a change in the gene, or a gene which regulates it, in the tissue of the human that was suspected of being diseased.

The subject nucleic acid and/or polypeptide compositions may be used to analyze a patient sample for the presence of polymorphisms associated with a disease state. Biochemical studies may be performed to determine whether a sequence polymorphism in a coding region or control regions is associated with disease, particularly cancers and other growth abnormalities. Diseases of interest may also include other hyperproliferative disorders. Disease associated polymorphisms may include deletion or truncation of the gene, mutations that alter expression level, that affect the binding activity of the protein, the kinase activity domain, etc.

Changes in the promoter or enhancer sequence that may affect expression levels of can be compared to expression levels of the normal allele by various methods known in the art. Methods for determining promoter or enhancer strength include quantitation of the expressed natural protein; insertion of the variant control element into a vector with a reporter gene such as beta-galactosidase, luciferase, chloramphenicol acetyltransferase, etc. that provides for convenient quantitation; and the like.

A number of methods are available for analyzing nucleic acids for the presence of a specific sequence, e.g. upregulated expression. Cells that express SEQ ID NO:1 may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki et al (1985) Science 239:487, and a review of techniques may be found in Sambrook, et al. Molecular Cloning: A Laboratory Manual, CSH Press 1989, pp. 14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis(DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

Arrays provide a high throughput technique that can assay a large number of polynucleotides in a sample. In one aspect of the invention, an array is constructed comprising one or more of SEQ ID NO:1, preferably comprising all of these sequences, which array may further comprise other sequences known to be up- or down-regulated in tumor cells. This technology can be used as a tool to test for differential expression.

A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of nucleic acids can be detectably labeled (e.g., using radioactive or fluorescent labels) and then hybridized to the probes. Double stranded nucleic acids, comprising the labeled sample polynucleotides bound to probe nucleic acids, can be detected once the unbound portion of the sample is washed away. Alternatively, the nucleic acids of the test sample can be immobilized on the array, and the probes detectably labeled.

Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of SEQ ID NO:1, where expression is compared between a test cell and control cell (e.g., cancer cells and normal cells). High expression of a particular message in a cancer cell, which is not observed in a corresponding normal cell, indicates a cancer specific gene product. Exemplary uses of arrays are further described in, for example, Pappalarado et al. (1998) Sem. Radiation Oncol. 8:217; and Ramsay. (1998) Nature Biotechnol. 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Polypeptide Analysis

Screening for expression of the subject sequences may be based on the functional or antigenic characteristics of the protein. Protein truncation assays are useful in detecting deletions that may affect the biological activity of the protein. Various immunoassays designed to detect polymorphisms in proteins encoded by SEQ ID NO:1 may be used in screening. Where many diverse genetic mutations lead to a particular disease phenotype, functional protein assays have proven to be effective screening tools. The activity of the encoded protein in kinase assays, etc., may be determined by comparison with the wild-type protein.

A sample is taken from a patient with cancer. Samples, as used herein, include biological fluids such as blood; organ or tissue culture derived fluids; etc. Biopsy samples or other sources of carcinoma cells are of particular interest, e.g. tumor biopsy, etc. Also included in the term are derivatives and fractions of such cells and fluids. The number of cells in a sample will generally be at least about 10³, usually at least 10⁴, and may be about 10⁵ or more. The cells may be dissociated, in the case of solid tissues, or tissue sections may be analyzed. Alternatively a lysate of the cells may be prepared.

Detection may utilize staining of cells or histological sections, performed in accordance with conventional methods. The antibodies or other specific binding members of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the cancer associated kinase corresponding to SEQ ID NO:1 in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind to one of the proteins encoded by SEQ ID NO:1 with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemilumninescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the cancer associated kinase corresponding to SEQ ID NO:1 as desired, conveniently using a labeling method as described for the sandwich assay.

In some cases, a competitive assay will be used. In addition to the patient sample, a competitor to the targeted protein is added to the reaction mix. The competitor and the cancer associated kinase corresponding to SEQ ID NO:1 compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

In some embodiments, the methods are adapted for use in vivo, e.g., to locate or identify sites where cancer cells are present. In these embodiments, a detectably-labeled moiety, e.g., an antibody, which is specific for the protein encoded by one of SEQ ID NO:1 is administered to an individual (e.g., by injection), and labeled cells are located using standard imaging techniques, including, but not limited to, magnetic resonance imaging, computed tomography scanning, and the like. In this manner, cancer cells are differentially labeled.

The detection methods can be provided as part of a kit. Thus, the invention further provides kits for detecting the presence of an mRNA corresponding to SEQ ID NO:1, and/or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

Samples for Analysis

Sample of interest include tumor tissue, e.g. excisions, biopsies, blood samples where the tumoris metastatic, etc. Of particular interest are solid tumors, e.g. carcinomas, and include, without limitation, tumors of the liver and colon. Liver cancers of interest include hepatocellular carcinoma (primary liver cancer). Also called hepatoma, this is the most common form of primary liver cancer. Chronic infection with hepatitis B and C increases the risk of developing this type of cancer. Other causes include cancer-causing substances, alcoholism, and chronic liver cirrhosis. Other liver cancers of interest for analysis by the subject methods include hepatocellular adenoma, which are benign tumors occuring most often in women of childbearing age; hemangioma, which are a type of benign tumor comprising a mass of abnormal blood vessels, cholangiocarcinoma, which originates in the lining of the bile channels in the liver or in the bile ducts; hepatoblastoma, which is common in infants and children; angiosarcoma, which is a rare cancer that originates in the blood vessels of the liver; and bile duct carcinoma and liver cysts. Cancers originating in the lung, breast, colon, pancreas and stomach and blood cells commonly are found in the liver after they become metastatic.

Also of interest are colon cancers. Types of polyps of the colon and rectum include polyps, which are any mass of tissue that arises from the bowel wall and protrudes into the lumen. Polyps may be sessile or pedunculated and vary considerably in size. Such lesions are classified histologically as tubular adenomas, tubulovillous adenomas (villoglandular polyps), villous (papillary) adenomas (with or without adenocarcinoma), hyperplastic polyps, hamartomas, juvenile polyps, polypoid carcinomas, pseudopolyps, lipomas, leiomyomas, or other rarer tumors.

Screening Methods

Target Screening. Reagents specific for SEQ ID NO:1 are used to identify targets of the encoded protein in tumor cells. For example, one of the nucleic acid coding sequences may be introduced into a tumor cell using an inducible expression system. Suitable positive and negative controls are included. Transient transfection assays, e.g. using adenovirus vectors, may be performed. The cell system allows a comparison of the pattern of gene expression in transformed cells with or without expression of the kinase. Alternatively, phosphorylation patterns after induction of expression are examined. Gene expression of putative target genes may be monitored by Northern blot or by probing microarrays of candidate genes with the test sample and a negative control where gene expression of the kinase is not induced. Patterns of phosphorylation may be monitored by incubation of the cells or lysate with labeled phosphate, followed by 1 or 2 dimensional protein gel analysis, and identification of the targets by MALDI, micro-sequencing, western blot analysis, etc., as known in the art.

Some of the potential target genes of the subject cancer associated kinase corresponding to SEQ ID NO:1 identified by this method will be secondary or tertiary in a complex cascade of gene expression or signaling. To identify primary targets of the subject kinase activation, expression or phosphorylation will be examined early after induction of expression (within 1-2 hours) or after blocking later steps in the cascade with cycloheximide.

Target genes or proteins identified by this method may be analyzed for expression in primary patient samples as well. The data for the subject cancer associated kinase corresponding to SEQ ID NO:1 and target gene expression may be analyzed using statistical analysis to establish a correlation.

Compound Screening. The availability of a number of components in signaling pathways allows in vitro reconstruction of the pathway, and/or assessent of kinase action on targets. Two or more of the components may be combined in vitro, and the behavior assessed in terms of activation of transcription of specific target sequences; modification of protein components, e.g. proteolytic processing, phosphorylation, methylation, etc.; ability of different protein components to bind to each other etc. The components may be modified by sequence deletion, substitution, etc. to determine the functional role of specific domains.

Compound screening may be performed using an in vitro model, a genetically altered cell or animal, or purified protein corresponding to any one of SEQ ID NO:1. One can identify ligands or substrates that bind to, modulate or mimic the action of the encoded polypeptide. Areas of investigation include the development of treatments for hyper-proliferative disorders, e.g. cancer, restenosis, osteoarthritis, metastasis, etc.

The polypeptides include those encoded by SEQ ID NO:1, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by SEQ ID NO:1, or a homolog thereof.

Transgenic animals or cells derived therefrom are also used in compound screening. Transgenic animals may be made through homologous recombination, where the normal locus corresponding to SEQ ID NO:1 is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in kinase activity, oncogenesis, signal transduction, etc. Of interest is the use of SEQ ID NO:1 to construct transgenic animal models for cancer, where expression of the corresponding kinase is specifically reduced or absent. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development. By providing expression of the target protein in cells in which it is not normally produced, one can induce changes in cell behavior, e.g. in the control of cell growth and tumorigenesis.

Compound screening identifies agents that modulate function of the cancer associated kinase corresponding to SEQ ID NO:1. Agents that mimic its function are predicted to activate the process of cell division and growth. Conversely, agents that inhibit function may inhibit transformation. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains, e.g. the kinase catalytic domain, the regulatory domain, the auto-inhibitory domain, etc.

The term “agent” as used herein describes any molecule, e.g. protein or pharmaceutical, with the capability of altering or mimicking the physiological function of a cancer associated kinase corresponding to SEQ ID NO:1. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Other assays of interest detect agents that mimic the function of a cancer associated kinase corresponding to SEQ ID NO:1. For example, an expression construct comprising the gene may be introduced into a cell line under conditions that allow expression. The level of kinase activity is determined by a functional assay, for example detection of protein phosphorylation. Alternatively, candidate agents are added to a cell that lacks the functional cancer associated kinase corresponding to SEQ ID NO:1, and screened for the ability to reproduce the activity in a functional assay.

The compounds having the desired pharmacological activity may be administered in a physiologically acceptable carrier to a host for treatment of cancer, etc. The compounds may also be used to enhance function in wound healing, cell growth, etc. The inhibitory agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-10 wt %.

Formulations. The compounds of this invention can be incorporated into a variety of formulations for therapeutic administration. Particularly, agents that modulate activity of a cancer associated kinase corresponding to SEQ ID NO:1, or polypeptides and analogs thereof are formulated for administration to patients for the treatment of cells where the target activity is undesirably high or low, e.g. to reduce the level of activity in cancer cells. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intra-tracheal, etc., administration. The agent may be systemic after administration or may be localized by the use of an implant that acts to retain the active dose at the site of implantation.

In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of disease, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic lipids, such as phosphatidylcholine. The remaining lipid will normally be neutral lipids, such as cholesterol, phosphatidyl serine, phosphatidyl glycerol, and the like.

Modulation of Enzyme Activity

Agents that block activity of cancer associated kinase corresponding to SEQ ID NO:1 provide a point of intervention in an important signaling pathway. Numerous agents are useful in reducing this activity, including agents that directly modulate expression as described above, e.g. expression vectors, antisense specific for the targeted kinase; and agents that act on the protein, e.g. specific antibodies and analogs thereof, small organic molecules that block catalytic activity, etc.

The genes, gene fragments, or the encoded protein or protein fragments are useful in therapy to treat disorders associated with defects in sequence or expression. From a therapeutic point of view, inhibiting activity has a therapeutic effect on a number of proliferative disorders, including inflammation, restenosis, and cancer. Inhibition is achieved in a number of ways. Antisense sequences may be administered to inhibit expression. Pseudo-substrate inhibitors, for example, a peptide that mimics a substrate for the kinase may be used to inhibit activity. Other inhibitors are identified by screening for biological activity in a functional assay, e.g. in vitro or in vivo kinase activity.

Expression vectors may be used to introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold micro projectiles are coated with the protein or DNA, then bombarded into skin cells.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Cancer Vaccines

MAK3K11 finds use in eliciting a immune response in an autologous, allogeneic or xenogeneic host. For example, where a tumor cell specifically expresses the protein, or over-expresses the protein relative to normal cells, a cytolytic immune response may be induced, where the tumor cell is preferentially killed. The antigen for such purposes may be from the same or a different species. As used herein, the term antigen is intended to refer to a molecule capable of eliciting an immune response in a mammalian host, which may be a humoral immune response, i.e. characterized by the production of antigen-specific antibodies, or a cytotoxic immune response, i.e. characterized by the production of antigen specific cytotoxic T lymphocytes.

The portion of the antigen bound by the antibody or T cell receptor is referred to as an epitope. Antigens, particular complex antigens such as polypeptides, usually comprise multiple epitopes. Where the antigen is a protein, linear epitopes range from about 5 to 20 amino acids in length. Antibodies and T cell receptor may also recognize conformational determinants formed by non-contiguous residues on an antigen, and an epitope can therefore require a larger fragment of the antigen to be present for binding, e.g. a protein domain, or substantially all of a protein sequence. It will therefore be appreciated that a therapeutic protein, which may be several hundred amino acids in length, can comprise a number of distinct epitopes.

Several methods exist which can be used to induce an immune response against weakly antigenic protein, i.e. autologous proteins, etc. The immunogen is usually delivered in vivo to elicit a response, but in some cases it is advantageous to prime antigen presenting cells, e.g. dendritic cells, ex vivo prior to introducing them into the host animal.

In the preparation of the antigen, a T_{BT} protein or a fragments thereof is expressed and purified as is known in the art. Alternatively, fragments of a T_{BT} protein may be chemically synthesized. In order to produce an immune response, the protein may be made as a fusion protein or otherwise conjugated to another polypeptide, and may be chemically modified or mixed with an adjuvant.

Examples of conjugates, which may utilize peptide linkage or other linkage to joint the molecules, include, for example KLH, pre-S HbsAg or cytokines or chemokines such as, for example interferon inducible protein 10 (IP-10), monocyte chemotactic protein 3 (MCP-3), interleukin-1,-2 and -8, granulocyte macrophage-colony stimulating factor (GM-CSF), etc, or may be chemically modified. Examples of suitable fusion chemokines and methods for antigen preparation and immunization are provided in Biragyn et al (Immunol Rev (1990) 170:115-126); Biragyn et al (Nature Biotechnology (1999) 17:253-258 and Tao et al Nature (1993) 362:755-695).

The polypeptide antigens may be mixed with an adjuvant that will augment specific immune reponses to the antigen. Many different types of adjuvants are known in the art and may include e.g. alum, stearyl tyrosine, saponin, monophosphoryl lipid A (MPL-A), muramyl tripeptide phosphatidylethanolamine (MTP-PE) etc. Adjuvants may also contain cytokines, such as interleukin 1 (IL1), interleukin 2 (IL2) other interleukins, TNFα, and γ-interferon, granulocyte macrophage-colony stimulating factor, tumor necrosis factor etc. Adjuvants may also contain other moieties such as cholera toxin B subunit, whole cell killed mycobacteria, Bordetella pertussis components, diptheria toxins and the like. Vaccine antigens may be presented using microspheres, liposomes, may be produced using an immunostimulating complex (ISCOM), as is known in the art.

Where an ex vivo antigen loading step is included, dendritic cells are isolated from an individual, using known methods, and incubated with the peptide antigen, preferably fused to a cytokine such as GM-CSF. The dendritic cell preparation may then be fractionated and administered to the host by intravenous or central injection according to established procedures (e.g., infusion over 30 to 60 minutes). The responsiveness of the subject to this treatment may measured by monitoring the induction of a cytolytic T-cell response, a helper T-cell response and antibody response towards the antigen in peripheral blood mononuclear cells by methods well known in the art. The disclosures of U.S. Pat. Nos. 5,851,756, 6,080,409, 5,994,126 and 5,972,334 are herein incorporated by reference in their entirety.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

MAP3K11 Sequence.

The Genbank database was searched for ESTs showing similarity to known kinase domain-related proteins using the “basic local alignment search tool” program, TBLASTN, with default settings. Human ESTs identified as having similarity to these known kinase domain (defined as p<0.0001) were used in a BLASTN and BLASTX screen of the GenBank non-redundant (NR) database.

ESTs that had top human hits with >95% identity over 100 amino acids were discarded. This was based upon the inventors' experience that these sequences were usually identical to the starting probe sequences, with the differences due to sequence error. The remaining BLASTN and BLASTX outputs for each EST were examined manually, i.e., ESTs were removed from the analysis if the inventors determined that the variation from the known kinase domain-related probe sequence was a result of poor database sequence. Poor database sequence was usually identified as a number of ‘N’ nucleotides in the database sequence for a BLASTN search and as a base deletion or insertion in the database sequence, resulting in a peptide frameshift, for a BLASTX output. ESTs for which the highest scoring match was to non-kinase domain-related sequences were also discarded at this stage.

Using widely known algorithms, e.g. “Smith/Waterman”, “Fasta”, “FastP”, “Needleman/Wunsch”, “Blast”, “PSIBlast,” homology of the subject nucleic acid to other known nucleic acids was determined. A “Local FastP Search” algorithm was performed in order to determine the homology of the subject nucleic acid invention to known sequences. Then, a ktup value, typically ranging from 1 to 3 and a segment length value, typically ranging from 20 to 200, were selected as parameters. Next, an array of position for the probe sequence was constructed in which the cells of the array contain a list of positions of that substring of length ktup. For each subsequence in the position array, the target sequence was matched and augmented the score array cell corresponding to the diagonal defined by the target position and the probe subsequence position. A list was then generated and sorted by score and report. The criterion for perfect matches and for mismatches was based on the statistics properties of that algorithm and that database, typically the values were: 98% or more match over 200 nucleotides would constitute a match; and any mismatch in 20 nucleotides would constitute a mismatch.

Analysis of the BLASTN and BLASTX outputs identified a EST sequence from IMAGE clone AI803752 that had potential for being associated with a sequence encoding a kinase domain-related protein, e.g., the sequence had homology, but not identity, to known kinase domain-related proteins.

After identification of MAP3K11 ESTs were discovered, the clones were added to Kinetek's clone bank for analysis of gene expression in tumor samples. Gene expression work involved construction of unigene clusters, which are represented by entries in the “pks” database. A list of accession numbers for members of the clusters were assigned. Subtraction of the clusters already present in the clone bank from the clusters recently added left a list of clusters that had not been previously represented in Kinetek's clone bank. For each of the clusters, a random selection of an EST IMAGE accession numbers were chosen to keep the clusters. For each of the clusters which did not have an EST IMAGE clone, generation of a report so that clone ordering or construction could be implemented was performed on a case by case basis. A list of accession numbers which were not in clusters was constructed and a report was generateds.

The AI803752 IMAGE clone was sequenced using standard ABI dye-primer and dye-terminator chemistry on a 377 automatic DNA sequencer. Sequencing revealed that the sequence corresponds to SEQ ID NO:1.

Rapid Amplification of cDNA Ends (RACE).

The gene specific oligodeoxynucleotide primers SEQ ID NO:3 and 4 were designed and then used to construct full length MAP3K11 cDNA by 5 prime RACE (rapid amplification of cDNA ends; Frohman et al. (1988), Proc. Natl. Acad. Sci. USA 85:8898-9002).

A nested primer strategy was used on human brain cDNA provided with a Marathon-Ready™ RACE kit (Clontech, Palo Alto, Calif.). Following this, thermal cycling on a PE DNA Thermal Cycler 480 was done. When cycling was completed, the PCR product was analyzed, along with appropriate DNA size markers, on a 1.0% agarose/EtBr gel.

The product so obtained comprised a MAP3K11 polynucleotide having the sequence of SEQ ID NO:1.

Expression Analysis of MAP3K11

The expression of MAP3K11 was determined by dot blot analysis, and the protein was found to be upregulated in several tumor samples.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human protein kinase arrays comprising human MAP3K11. The amount of radiolabeled probe hybridized to each arrayed EST clone was detected using phosphorimaging.

The expression of MAP3K11 was substantially upregulated in the tumor tissues that were tested. The data is shown in Table 1, expressed as the fold increase over the control non-tumor sample.

TABLE 1

| | liver | liver | liver | colon | colon | colon | colon | colon | colon | colon |
|---|---|---|---|---|---|---|---|---|---|---|
| Target | 1 | 2 | 3 | 1 | 4 | 5 | 7 | 8 | 9 | 10 |
| MAP3K11 | 4.1 | 1.3 | 2.3 | 2.1 | 1.1 | 1.9 | 3.4 | 1.3 | 0.9 | 1.75 |
| beta-actin | 2.05 | 1.07 | 1.57 | 0.42 | 1.28 | 2.19 | 1.20 | 4.60 | 0.60 | 0.49 |
| GAPDH | 1.30 | 0.33 | 1.25 | 0.76 | | | | | | |
| K413 | | | | | 1.72 | 2.36 | 2.10 | 1.00 | 1.00 | 1.68 |
| (ribosomal | | | | | | | | | | |
| protein) | | | | | | | | | | |

The data displayed in Table 2 provides a brief summary of the pathology report of the patient samples.

TABLE 2

| | | | Precursor | Site of | | Vascular | Lymphatic | |
|---|---|---|---|---|---|---|---|---|
| Patient | Age | Gender | Adenoma | Involvement | Differentiation | Invasion | Involvement | Metastasis |
| Liver 1 | 49 | Female | N/a | Liver | Moderately | No | Yes | No |
| | | | | | Differentiated | | | |
| Liver 2 | 53 | Male | N/a | Liver | Moderately | Yes | No | No |
| | | | | | Differentiated | | | |
| Liver 3 | 75 | Female | Adenoma | Right Colon | Moderately | No | No | No |
| | | | | | differentiated | | | |
| Colon 1 | 55 | Female | No | Rectum | Moderately | N/A | Yes | No |
| | | | | | Differentiated | | | |
| Colon 4 | 91 | Female | Adenoma | Cecum | Moderately | No | Yes | No |
| | | | | | Differentiated | | | |
| Colon 5 | 79 | Male | No | Ileum and | | | | |
| | | | | Colon | | | | |
| Colon 7 | | | | | Moderately | No | No | No |
| | | | | | Differentiated | | | |
| Colon 8 | 61 | Male | Yes | | Moderately | No | Yes | Yes, Liver |
| | | | | | Differentiated | | | |
| Colon 9 | 60 | Male | No | Recto- | Moderately | Yes | No | Yes, Liver |
| | | | | Sigmoid | Differentiated | | | |
| Colon 10 | 60 | Male | No | Sigmoid | Moderately | Yes | Yes | No |
| | | | | Colon | Differentiated | | | |

What is claimed is:

1. A method of screening for biologically active agents that modulate a cancer associated protein kinase function, the method comprising:combining a candidate biologically active agent with any one of:
(a) a polypeptide encoded by any one of SEQ ID NO:1, 3, 5, 7, 9, 11 or 13;
(b) a cell comprising a nucleic acid encoding a polypeptide encoded by any one of SEQ ID NO:1, 3, 5, 7, 9, 11 or 13; or
(c) a non-human transgenic animal model for cancer associated kinase gene function comprising one of: (i) a knockout of a gene corresponding any one of SEQ ID NO:1, 3, 5, 7, 9, 11 or 13; (ii) an exogenous and stably transmitted mammalian gene sequence comprising polypeptide encoded by any one of SEQ ID NO:1, 3, 5, 7, 9, 11 or 13; and
determining the effect of said agent on kinase function.

2. A method for the diagnosis of cancer, the method comprising:
determining the upregulation of expression in any one of SEQ ID NO:1, 3, 5, 7, 9, 11 or 13 in said cancer.

3. The method of claim 2, wherein said cancer is a liver cancer.

4. The method of claim 2, wherein said cancer is a colon cancer.

5. The method of claim 2, wherein said determining comprises detecting the presence of increased amounts of mRNA in said cancer.

6. The method of claim 2, wherein said determining comprises detecting the presence of increased amounts of protein in said cancer.

7. A method for inhibiting the growth of a cancer cell, the method comprising downregulating activity of the polypeptide encoded by any one of SEQ ID NO: 1, 3, 5, 7, 9, 11 or 13 in said cancer cell.

8. The method according to claim 7, wherein said method comprises introducing antisense sequences specific for any one of SEQ ID NO:1, 3, 5, 7, 9, 11 or 13.

9. The method according to claim 7, wherein said method comprises introducing an inhibitor of kinase activity into said cancer cell.

10. The method according to claim 7, wherein said cancer cell is a liver cancer cell.

11. The method according to claim 7, wherein said cancer cell is a colon cancer cell.

12. A method of screening for targets of a cancer associated protein kinase, wherein said targets are associated with signal transduction in cancer cells, the method comprising:
comparing the pattern of gene expression in a normal cell, and in a tumor cell characterized by up-regulation of any one of SEQ ID NO:1, 3, 5, 7, 9, 11 or 13.

13. The method according to claim 12, wherein said comparing the pattern of gene expression comprises quantitating specific mRNAs by hybridization to an array of polynucleotide probes.

14. A method of screening for targets of a cancer associated protein kinase, wherein said targets are associated with signal transduction in cancer cells, the method comprising:
comparing the pattern of protein phosphorylation in a normal cell, and in a tumor cell characterized by up-regulation of any one of SEQ ID NO:1, 3, 5, 7, 9, 11 or 13.

15. An isolated nucleic acid comprising the sequence set forth in any one of SEQ ID NO:1, 3, 5, 7, 9, 11 or 13.


SEQUENCE LISTING (4 sequences)

SEQ ID NO: 1 - DNA, 3558 bp, Homo sapien
  CDS (482)...(3023)
ggaagaagggagcggggtcggagccgtcggggccaaaggagacggggccaggaacaggcagtctcggcccaactgcggacgctccctccaccccctgcgcaaaaagacccaaccggagttgaggcgctgcccctgaaggccccaccttacacttggcgggggccggagccaggctcccaggactgctccagaaccgagggaagctcgggtccctccaagctagccatggtgaggcgccggaggccccggggccccacccccccggcctgaccacactgccctgggtgccctcctccagaagcccgagatgcggggggccgggagacaacactcctggctccccagagaggcgtgggtctggggctgagggccagggcccggatgcccaggttccgggactagggccttggcagccagcgggggtggggaccacgggcacccagagaaggtcctccacacatcccagcgccggctcccggccatggagcccttgaagagcctcttcctcaagagccctctagggtcatggaatggcagtggcagcgggggtggtgggggcggtggaggaggccggcctgaggggtctccaaaggcagcgggttatgccaacccggtgtggacagccctgttcgactacgagcccagtgggcaggatgagctggccctgaggaagggtgaccgtgtggaggtgctgtcccgggacgcagccatctcaggagacgagggctggtgggcgggccaggtgggtggccaggtgggcatcttcccgtccaactatgtgtctcggggtggcggcccgcccccctgcgaggtggccagcttccaggagctgcggctggaggaggtgatcggcattggaggctttggcaaggtgtacaggggcagctggcgaggtgagctggtggctgtgaaggcagctcgccaggaccccgatgaggacatcagtgtgacagccgagagcgttcgccaggaggcccggctcttcgccatgctggcacaccccaacatcattgccctcaaggctgtgtgcctggaggagcccaacctgtgcctggtgatggagtatgcagccggtgggcccctcagccgagctctggccgggcggcgcgtgcctccccatgtgctggtcaactgggctgtgcagattgcccgtgggatgcactacctgcactgcgaggccctggtgcccgtcatccaccgtgatctcaagtccaacaacattttgctgctgcagcccattgagagtgacgacatggagcacaagaccctgaagatcaccgactttggcctggcccgagagtggcacaaaaccacacaaatgagtgccgcgggcacctacgcctggatggctcctgaggttatcaaggcctccaccttctctaagggcagtgacgtctggagttttggggtgctgctgtgggaactgctgaccggggaggtgccataccgtggcattgactgccttgctgtggcctatggcgtagctgttaacaagctcacactgcccatcccatccacctgccccgagcccttcgcacagcttatggccgactgctgggcgcaggacccccaccgcaggcccgacttcgcctccatcctgcagcagttggaggcgctggaggcacaggtcctacgggaaatgccgcgggactccttccattccatgcaggaaggctggaagcgcgagatccagggtctcttcgacgagctgcgagccaaggaaaaggaactactgagccgcgaggaggagctgacgcgagcggcgcgcgagcagcggtcacaggcggagcagctgcggcggcgcgagcacctgctggcccagtgggagctagaggtgttcgagcgcgagctgacgctgctgctgcagcaggtggaccgcgagcgaccgcacgtgcgccgccgccgcgggacattcaagcgcagcaagctccgggcgcgcgacggcggcgagcgtatcagcatgccactcgacttcaagcaccgcatcaccgtgcaggcctcacccggccttgaccggaggagaaacgtcttcgaggtcgggcctggggattcgcccacctttccccggttccgagccatccagttggagcctgcagagccaggccaggcatggggccgccagtccccccgacgtctggaggactcaagcaatggagagcggcgagcatgctgggcttggggtcccagttcccccaagcctggggaagcccagaatgggaggagaaggtcccgcatggacgaagccacatggtacctggattcagatgactcatcccccttaggatctccttccacacccccagcactcaatggtaaccccccgcggcctagcctggagcccgaggagcccaagaggcctgtccccgcagagcgcggtagcagctctgggacgcccaagctgatccagcgggcgctgctgcgcggcaccgccctgctcgcctcgctgggccttggccgcgacctgcagccgccgggaggcccaggacgcgagcgcggggagtccccgacaacaccccccacgccaacgcccgcgccctgcccgaccgagccgcccccttccccgctcatctgcttctcgctcaagacgcccgactccccgcccactcctgcacccctgttgctggacctgggtatccctgtgggccagcggtcagccaagagcccccgacgtgaggaggagccccgcggaggcactgtctcacccccaccggggacatcacgctctgctcctggcaccccaggcaccccacgttcaccacccctgggcctcatcagccgacctcggccctcgccccttcgcagccgcattgatccctggagctttgtgtcagctgggccacggccttctcccctgccatcaccacagcctgcaccccgccgagcaccctggaccttgttcccggactcagaccccttctgggactccccacctgccaaccccttccaggggggcccccaggactgcagggcacagaccaaagacatgggtgcccaggccccgtgggtgccggaagcggggccttgagtgggccaggccactcccccgagctccagctgccttaggaggagtcacagcatacactggaacaggagctgggtcagcctctgcagctgcctcagtttccccagggaccccacccccctttgggggtcaggaacactacactgcacaggaagccttcacactggaagggggacctgcgcccccacatctgaaacctgtaggtccccccagctcacctgccctactggggcccaacactgtacccagctggttgggaggaccagagcctgtctcagggaattgcctgctggggtgatgcagggaggaggggaggtgcagggaagaggggccggcctcagctgtcaccagcacttttgaccaagtcctgctactgcggcccctgccctagggcttagagcatggacctcctgccctgggggtcatctggggccagggctctctggatgccttcctgctgccccagccagggttggagtcttagcctcgggatccagtgaagccagaagccaaataaactcaaaagctgtctcccc

SEQ ID NO: 2 - protein, 847 aa, Homo sapien
MEPLKSLFLKSPLGSWNGSGSGGGGGGGGGRPEGSPKAAGYANPVWTALFDYEPSGQDELALRKGDRVEVLSRDAAISGDEGWWAGQVGGQVGIFPSNYVSRGGGPPPCEVASFQELRLEEVIGIGGFGKVYRGSWRGELVAVKAARQDPDEDISVTAESVRQEARLFAMLAHPNIIALKAVCLEEPNLCLVMEYAAGGPLSRALAGRRVPPHVLVNWAVQIARGMHYLHCEALVPVIHRDLKSNNILLLQPIESDDMEHKTLKITDFGLAREWHKTTQMSAAGTYAWMAPEVIKASTFSKGSDVWSFGVLLWELLTGEVPYRGIDCLAVAYGVAVNKLTLPIPSTCPEPFAQLMADCWAQDPHRRPDFASILQQLEALEAQVLREMPRDSFHSMQEGWKREIQGLFDELRAKEKELLSREEELTRAAREQRSQAEQLRRREHLLAQWELEVFERELTLLLQQVDRERPHVRRRRGTFKRSKLRARDGGERISMPLDFKHRITVQASPGLDRRRNVFEVGPGDSPTFPRFRAIQLEPAEPGQAWGRQSPRRLEDSSNGERRACWAWGPSSPKPGEAQNGRRRSRMDEATWYLDSDDSSPLGSPSTPPALNGNPPRPSLEPEEPKRPVPAERGSSSGTPKLIQRALLRGTALLASLGLGRDLQPPGGPGRERGESPTTPPTPTPAPCPTEPPPSPLICFSLKTPDSPPTPAPLLLDLGIPVGQRSAKSPRREEEPRGGTVSPPPGTSRSAPGTPGTPRSPPLGLISRPRPSPLRSRIDPWSFVSAGPRPSPLPSPQPAPRRAPWTLFPDSDPFWDSPPANPFQGGPQDCRAQTKDMGAQAPWVPEAGP

SEQ ID NO: 3 - DNA, 28 bp, Primer sequence 1
gaattccatggagcccttgaagagcctc

SEQ ID NO: 4 - DNA, 28 bp, Primer sequence 2
ctcgagtcaaggccccgcttccggcacc